(19) United States
(12) Patent Application Publication
(10) Pub. No.: US 2001/0014457 A1
(43) Pub. Date: Aug. 16, 2001

(54) NUCLEIC ACIDS ENCODING RECEPTORS FOR BOMBESIN-LIKE PEPTIDES

(76) Inventors: ELIOT R. SPINDEL, LAKE OSWEGOR, OR (US); SRINIVASA NAGALLA, PORTLAND, OR (US); BRENDA BARRY, PORTLAND, OR (US)

Correspondence Address: JOHN D CONWAY, BIOMEASURE INC, 27 MAPLE STREET, MILFORD, MA, 01757

(21) Appl. No.: 09/160,116

(22) Filed: Sep. 24, 1998

Related U.S. Application Data
(63) Continuation-in-part of application No. 08/910,092, filed on Aug. 12, 1997, now Pat. No. 5,814,463.
Application No. 08/910,092 is a division of application No. 08/279,590, filed on Jul. 22, 1994, now Pat. No. 5,656,749.

(51) Int. Cl. C12Q 1/68; G01N 33/53

(52) U.S. Cl. 435/7.2; 536/23.5; 435/320.1; 435/325; 435/6; 530/324; 530/387.9

(57) ABSTRACT

Pure nucleic acids encoding novel receptors for bombesin-like peptides, the novel receptors themselves, and their antibodies. Also disclosed is a method of screening for a compound capable of interacting with any of these novel receptors.

### CROSS REFERENCE TO RELATED APPLICATION

This application is a continuation-in-part of application Ser. No. 08/910,092, filed Aug. 12, 1997, now issued as U.S. Pat. No. 5,814,463, which is a divisional application of application Ser. No. 08/279,590, filed Jul. 22, 1994, now issued as U.S. Pat. No. 5,656,749.

### STATEMENT AS TO FEDERALLY SPONSORED RESEARCH

This invention was made with support from the National Institute of Health (Grant No. R01-CA39237). Accordingly, the U.S. government may have certain rights in the invention.

### FIELD OF THE INVENTION

The present invention relates generally to the manipulation of genetic materials, and, more particularly, to recombinant DNA procedures which make possible the identification of novel DNA sequences and polypeptides encoded thereby.

### BACKGROUND OF THE INVENTION

Bombesin, a tetradecapeptide amide first isolated from the skin of the frog Bombina bombina, is a potent mitogen for mouse Swiss 3T3 fibroblast cells. It also stimulates secretion for guinea pig pancreatic acini. Bombesin-like peptides are produced and secreted by human small cell lung cancer cells and exogenously added bombesin-like peptides can stimulate the growth of human SCLC cells in vitro. Two examples of bombesin-like peptides are gastrin releasing peptide (GRP) and neuromedin B (NMB).

GRP is a 27 amino acid peptide amide and was first isolated from the porcine gut. The C-terminal amino acid sequence of GRP is almost identical to that of bombesin. NMB, on the other hand, is a decapeptide amide, the structure of which is almost identical to the last ten amino acids in the C-terminal region of GRP. Both GRP and NMB share high amino acid sequence homology with bombesin and indeed possess bombesin-like properties. Other bombesin-like peptides include litorin and neuromedin C (NMC).

Recent structure-function and DNA cloning studies demonstrate that at least two classes of receptors mediate the action of bombesin-like peptides. One class, the GRP preferring subtype (GRP receptor), has a high affinity for GRP and a low affinity for NMB, whereas the other class, the NMB-preferring subtype (NMB receptor), has a high affinity for NMB and lower affinity for GRP. Both classes of receptors are widely present both in the central nervous system and in the gastrointestinal tract. A third receptor class, the BRS-3 receptor, has recently been found in both rat testes and pregnant uteruses. Unlike the GRP and NMB receptors, none of the presently known bombesin-like peptide binds with high affinity (K_{d}<25 nM) to the BRS-3 receptor.

### SUMMARY OF THE INVENTION

We have discovered novel genes which code for receptors capable of binding to bombesin-like peptides. The term “bombesin-like peptide” used here and below refers to a peptide capable of binding with a K_{d} less than 1 μM to either the GRP receptor, the NMB receptor, the BRS-3 receptor, or to any other bombesin receptor subtypes such as the BB4 and BB5 receptors described below. Examples of bombesin-like peptides include, but are not limited to, bombesin, GRP, NMB, NMC, BB4 and BB5.

Accordingly, in one aspect, the invention features a pure nucleic acid (for example, genomic DNA, cDNA, or RNA) encoding a receptor for a bombesin-like peptide, the receptor including SEQ ID NO: 2, SEQ ID NO: 4, SEQ ID NO: 6 or SEQ ID NO: 12 (e.g., either as the entirety of the receptor or as a fragment thereof). In other words, a pure nucleic acid which encodes a receptor for a bombesin-like peptide and includes SEQ ID NO: 1, SEQ ID NO: 3, SEQ ID NO: 5 or SEQ ID NO: 11; or a degenerate variant thereof embodies an aspect of this invention.

The invention also features a pure nucleic acid which (i) is capable of hybridizing to SEQ ID NO: 1, SEQ ID NO: 3, or SEQ ID NO: 5 under a high- or a low-stringency hybridization condition; and (ii) encodes a receptor protein for a bombesin-like peptide. By “low-stringency hybridization condition” is meant: prehybridization in 25% formamide, 5×SSC, 25 mM potassium phosphate buffer (pH 7.4), 5×Denhardt's, and 50 μg/ml denatured salmon sperm DNA for 4-12 hours at 37° C., which is followed by hybridization for 12-24 hours at 37° C. and washing in 2×SSC containing 0.1% SDS, at 42° C.; or an equivalent thereof. By “high-stringency hybridization condition” is meant: prehybridization in 50% formamide, 5×SSC, 25 mM potassium phosphate buffer (pH 7.4), 5×Denhardt's, and 50 μg/ml denatured salmon sperm DNA for 4-12 hours at 37° C., which is followed by hybridization for 12-24 hours at 37° C. and washing in 2×SSC containing 0.1% SDS, at 55° C.; or an equivalent thereof. E.g., see Sambrook, et al. Molecular Cloning, A Laboratory Manual, 2nd Ed. Cold Spring Harbor Laboratory Press, New York (1989), the contents of which are hereby incorporated by reference.

In another aspect, this invention is directed to a pure nucleic acid which is capable of hybridizing to SEQ ID NO. 11 under a high- or a low-stringency hybridization condition (as described above); and encodes a receptor protein for a bombesin-like peptide.

In related aspects, a cell containing one of the nucleic acids mentioned above, and a vector which includes such a nucleic acid and is capable of directing expression of the peptide encoded by that nucleic acid in a vector-containing cell are also within the scope of this invention.

Other embodiments include a pure receptor protein encoded by a nucleic acid of this invention which is capable of binding to a bombesin-like peptide, and a pure antibody which is specific for such a receptor protein.

In another aspect, this invention features a method of screening for a compound capable of interacting with a receptor protein for a bombesin-like peptide, the method comprising the steps of: (i) providing a cell which expresses a receptor protein of this invention (e.g., a native cell expressing the receptor obtained from the brain tissue, a frog egg into which mRNA encoding the receptor is introduced, or a host cells into which DNA encoding the receptor protein is introduced for expression); (ii) contacting the compound with the receptor protein expressed by the cell; and (iii) detecting an interaction, if any, between the compound and the receptor protein (e.g., binding or any biochemical response as a result of the binding).

By “pure nucleic acid” is meant a nucleic acid that is free or substantially free (i.e., at least 60% by weight free) of the DNA or RNA sequences which, in the naturally-occurring genome of the organism from which the nucleic acid of the invention is derived, flank it. The term therefore includes, for example, a recombinant DNA which is incorporated into a vector, into an autonomously replicating plasmid or virus, or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence. Chemically synthesized nucleic acids are also encompassed.

By “protein” is meant any chain of amino acids, regardless of length or post-translational modification (e.g., glycosylation or phosphorylation).

By “pure receptor protein” or “pure antibody” is meant a receptor protein or antibody which has been substantially separated from components which naturally accompany it, i.e., it is at least 60%, by weight, free from the proteins and naturally-occurring organic molecules with which it is naturally associated. A pure protein (i.e., a receptor protein or an antibody of this invention) may be obtained, for example, by extraction from a natural source, by expression of a recombinant nucleic acid, or by chemical synthesis. Purity can be measured by any appropriate method, e.g., those described in column chromatography, polyacrylamide gel electrophoresis, or by HPLC analysis.

Other features and advantages of the invention will be apparent from the following description and from the claims.

### BRIEF DESCRIPTION OF THE DRAWINGS

The drawings are first briefly described.

FIGS. 1A, 1B and 1C show a nucleotide sequence encoding the frog BB4 receptor; the encoded amino acid sequence is also shown.

FIG. 2 is a graph showing the responses to exogenous bombesin of Xenopus oocytes injected respectively with RNA's encoding the human GRP receptor and the frog BB4 receptor.

FIG. 3 is the encoded amino acid sequence of rat BRS-3 receptor aligned with the amino acid sequence of human BRS receptor.

FIG. 4 is the encoded amino acid sequence of the rat BRS-3 receptor aligned with the amino acid sequence of the frog BB4 receptor.

FIG. 5 shows an alignment and sequence conservation of the Bombesin family of receptors, the rat BRS-3, frog BB4, human BRS, human GRP receptor and human neuromedin B (NMB) receptor.

FIG. 6 shows a hydropathy analysis of the rat BRS-3 receptor sequence, demonstrating the seven transmembrane regions in the predicted topology of this class of receptors.

### DETAILED DESCRIPTION OF THE INVENTION

Insertion of a DNA sequence of this invention into a vector, introduction of the recombinant vector thus obtained into a host cell, and subsequent expression of a receptor protein encoded by the inserted DNA sequence can be performed to produce that receptor protein. Such techniques, which are well known to a person of ordinary skill in the art, can be found in the literature, e.g., Sambrook, et al. Molecular Cloning, A Laboratory Manual, 2nd Ed. Cold Spring Harbor Laboratory Press, New York (1989), the contents of which are hereby incorporated by reference. Note that all nucleic acid sequences of this invention can be readily prepared by a person of ordinary skill in the art employing one or more of the DNA sequences disclosed herein.

A receptor of this invention or its fragment (produced recombinantly, synthetically, or by conventional purification methods) can be used to generate an antibody (monoclonal or polyclonal) to be used as a diagnostic tool for detecting that receptor on cells from a given tissue, since the presence or expression level of that receptor may be related to cancer or other disorders. Of course, such an antibody can also be generated using a peptide fragment (e.g., a fragment of that receptor) which has at least one antigenic determinant that is immunologically reactive with an antigenic determinant of that receptor. Methods of generating and collecting such an antibody are well known in the art. For example, see Harlow et al., Antibodies— Laboratory Manual (1988, Cold Spring Harbor Laboratory), the contents of which are hereby incorporated by reference.

Conversely, any positively identified cells can be used to screen for compounds (e.g., a synthetic compound or the native ligand of that receptor) which interact with that receptor in various ways. As an example, bombesin-like peptides are produced and secreted by human small cell lung cancer cells (see BACKGROUND OF THE INVENTION above). Thus, some of the positively identified compounds (agonists or antagonists) can be used in the diagnosis or treatment of small cell lung cancer.

One way of detecting an interaction between a compound and the receptor of this invention is to monitor changes in intracellular calcium, as demonstrated in an actual example shown below. Alternatively, binding assays can be performed in screening for compounds which interact with the receptor. For experimental details, see von Schrenck T., et al. Am. J. Physiol. 1989; 256:G747-G758; and Moody T. W., et al., Methods Enzymol. 1989; 168:481-493, the contents of both of which are hereby incorporated by reference.

Without further elaboration, it is believed that one skilled in the art can, based on the description herein, utilize the present invention to its fullest extent. The following specific embodiments are, therefore, to be construed as merely illustrative, and not limitative of the remainder of the disclosure in any way whatsoever.

Identification of the Rat BRS-3 Receptor

The cDNA encoding the rat BRS-3 receptor was identified by screening a rat brain cDNA library (purchased from Clontech, Inc., Palo Alto, Calif.) at low stringency (25% formamide, 5×SSC, 37° C. with washing at 50° C. in 2× SSC) using the frog BB4 sequence (SEQ ID No. 1) as a probe using the method described in Sambrook, et al., Molecular Cloning, A Laboratory Manual, 2^{nd} Edition, Cold Spring Harbor Laboratory Press, NY, 1989, the contents of which are incorporated herein by reference. The frog BB4 sequence (SEQ ID No. 1) was ³²P-labeled using the nick-translation method described in Sambrook, et al. Multiple hybridizing clones were identified. DNA sequence analysis of one of the clones revealed a 2595 nucleotide sequence (SEQ ID NO: 11) encoding the full length 399 amino acid residues (SEQ ID NO: 12) of the rat BRS-3 receptor.

Identification of the Rat BRS-3 Receptor to the Bombesin-Like Family of Receptors

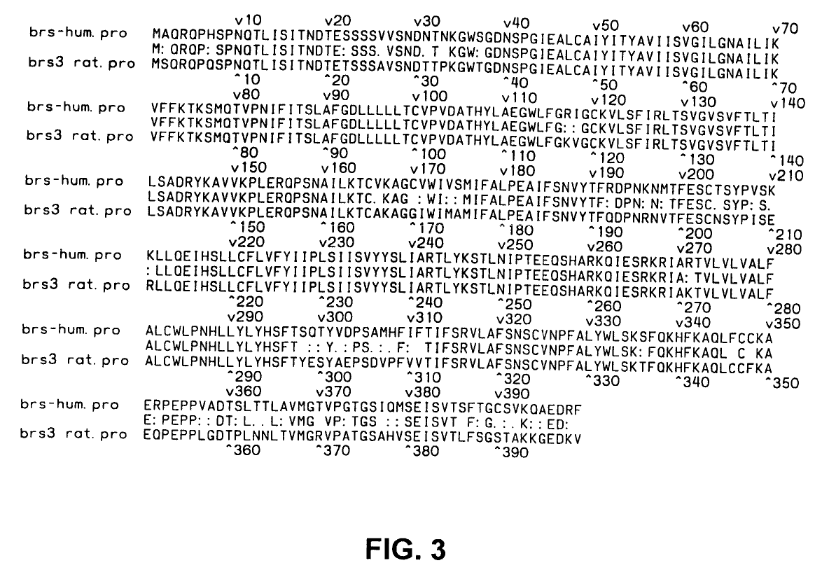

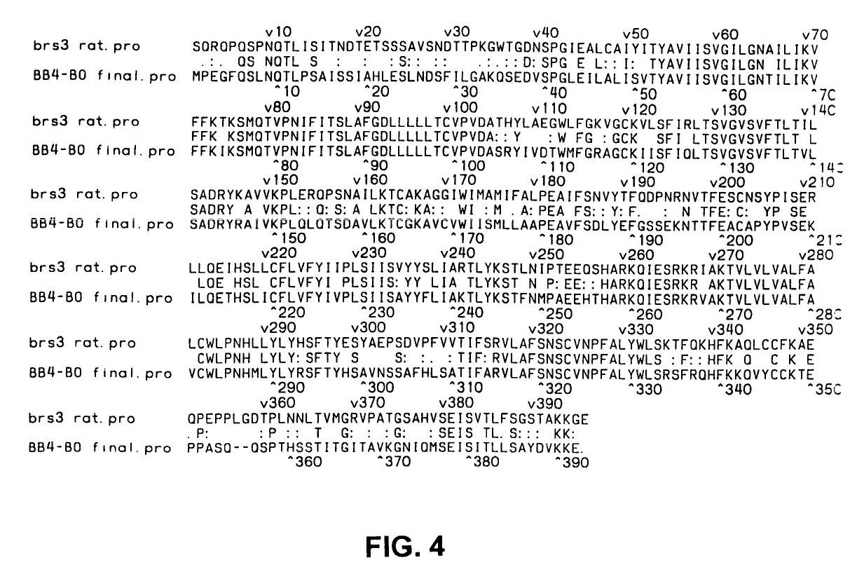

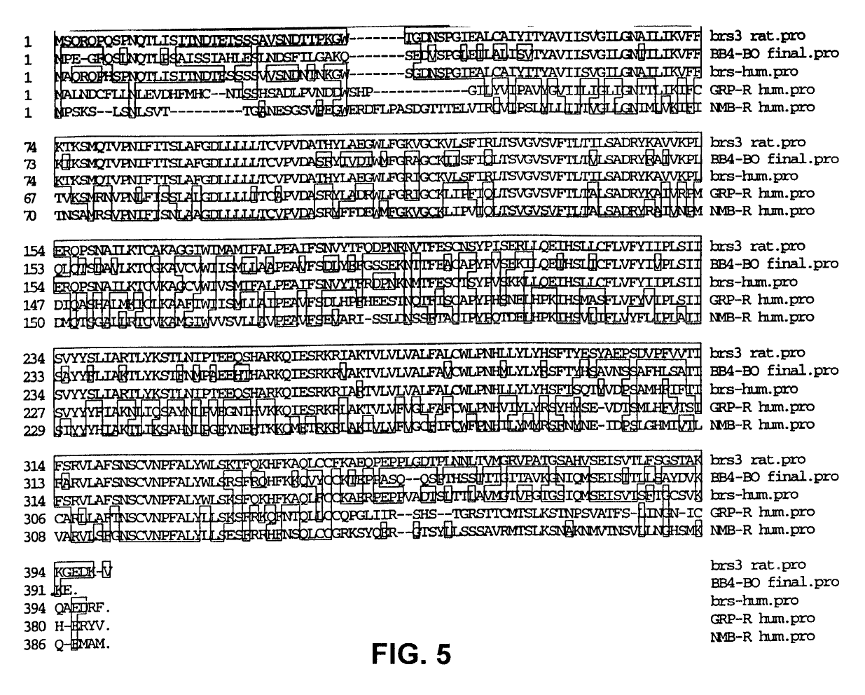

The rat BRS-3, frog BB4, human BRS (Fathi, Z., et al., J. Biol. Chem., 1993, 268, 5979-5984, the contents of which are incorporated herein by reference), GRP and NMB receptors were aligned in a manner described in Spindel, et al., Recent Prog. Horm. Res. 1993; 48:365, 380-381, the contents of which are hereby incorporated by reference. The aligned sequence in FIG. 4 showed that rat BRS-3 receptor shares a 64% amino acid identity to the frog BB4 receptor, an 86% amino acid identity with the human BRS receptor (FIG. 3) and 47% and 45% amino acid identity to the human GRP and NMB receptors, respectively (FIG. 5). This result suggests that this new receptor designated as rat BRS-3 receptor, is a member of the Bombesin-like peptide family of receptors. Its high homology (>80%) with the human BRS receptor suggests that the sequence is a rat homologue of the human BRS receptor.

Identification of Novel Receptors for Bombesin-Like Peptides

The rat, mouse, and human GRP receptors and the human and rat NMB receptors were aligned in a manner described in Spindel, et al., Recent Prog. Horm. Res. 1993; 48:365, 380-81, the contents of which are hereby incorporated by reference. This multiple alignment indicated certain conserved regions, based on which PCR primers and probes were prepared as tools used to look for novel receptors for bombesin-like peptides. More specifically, the following primers/probes were prepared: AT (ACT) CA(AG) CTI ACI TCI GTI GGI GTI TCI GT (SEQ ID NO: 7); (GA)TA IAG IGC (GA)AA IGG (AG)TT IAC (GA)CA IGA (GA)TT (SEQ ID NO: 8); (AC)G(ACGT) AA(AG) (AC)G(ACGT) (CT)T(ACGT) GC(ACGT) AA (SEQ ID NO: 9); and CC(ACGT) AC(GA) AA(ACGT) AC(ACGT) A(GA)(ACGT) AC (SEQ ID NO: 10). All primers/probes are written 5′ to 3′, mixed residues are shown in parentheses, and the symbol “I” denotes deoxyinosine.

Total RNA was then prepared by homogenization of frog brain (Bombina orientalis) in guanidine thiocyanate followed by centrifugation through CsCl. 5 μg total RNA was reverse transcribed with 25 pmole oligo(dT¹⁸), 200 units of M-MLV reverse transcriptase (GIBCO-BRL, Gaithersburg, Md.), 5× buffer (250 mM Tris-HCl, pH 8.3; 375 mM KCl, 15 mM MgCl₂, 50 mM DTT, 2.5 mM dNTP's) in 20 μl total volume at 37° C. for 1 hour. The entire reverse transcription was used in a 100 μl PCR reaction using 100 pmoles of SEQ ID NO: 7 and 100 pmoles of SEQ ID NO: 8. PCR conditions consisted of one cycle at 92° C.×2 min, 55° C.×2 min, 72° C.×3 min for second strand synthesis, followed by 35 cycles of 92° C.×40 sec, 55° C.×1 min, 72° C.×2 min. A 20 μl-aliquot of this reaction was separated on a 1% agarose gel, transferred to a Nylon membrane and hybridized to two ³²P-end labeled internal oligonucleotide probes (SEQ ID NO: 9 and SEQ ID NO: 10). The hybridizing product was subcloned into PGEM-T vector (Promega, Madison, Wis.) and sequenced as described in Nagalla, et al., J. Biol. Chem. 1992; 267:6916-22, which is hereby incorporated by reference.

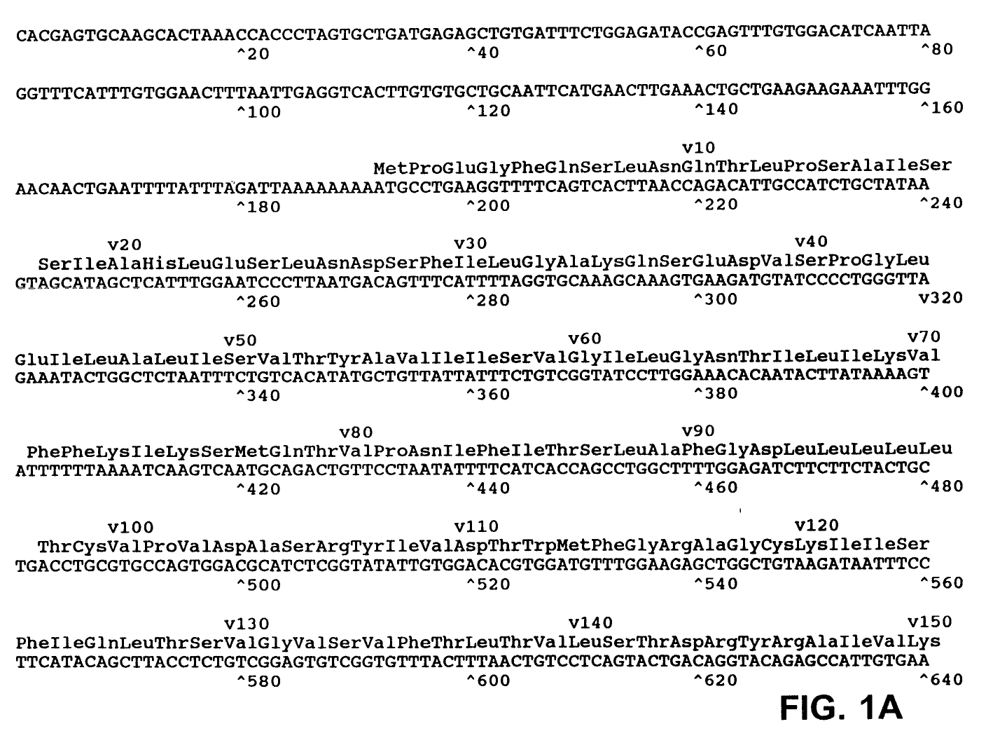

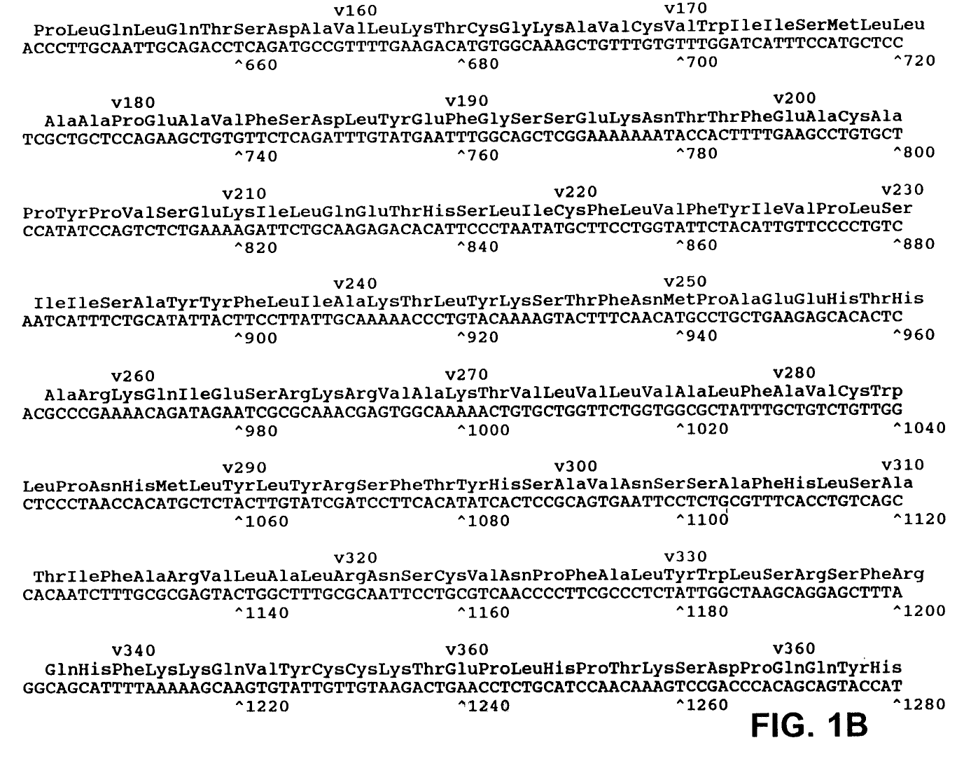

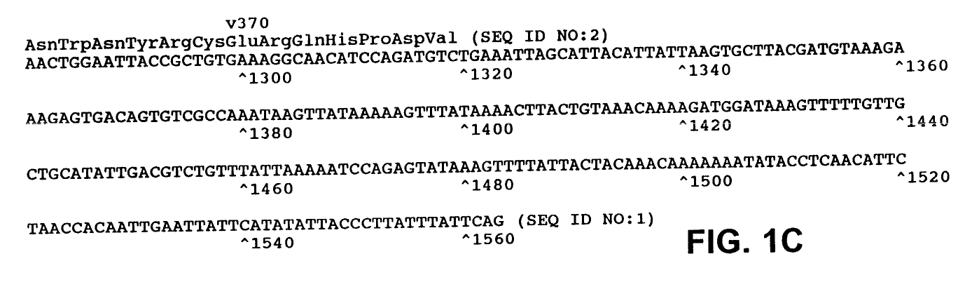

Sequence analysis of multiple clones revealed a nucleotide sequence corresponding to position 585-position 1178 of SEQ ID NO: 1, which encoded amino acid sequence corresponding to position 132-position 329 of SEQ ID NO: 2. Both SEQ ID NO: 1 and 2 are shown in FIG. 1. The homology of the encoded amino acid sequence with the GRP, NMB and BRS-3 receptors was analyzed. The encoded amino acid sequence showed a 70.7% homology with the BRS-3 receptor, a 61.1 % homology with the GRP receptor, and a 51.1% homology with the NMB receptor. These results suggested that this newly discovered encoded amino acid sequence represented a new receptor subtype, which is designated as frog BB4 receptor. To prove that this receptor was not the GRP or NMB receptor, other clones were isolated from frog stomach, brain and skin mRNA that had higher than 80% homology with their mammalian counterparts.

A cDNA library was next constructed from B. orientalis brain in the vector λZAP II (Stratagene, Inc., La Jolla, Calif.) using reagents and protocols provided by the supplier. To screen the cDNA library, a ³²P-labeled cRNA probe was prepared from the nucleotide sequence which corresponds to position 585-position 1178 of SEQ ID NO: 1 using the T7 promoter in the PGEM-T vector. A hybridizing clone was isolated and found to encode the full coding sequence of the Bombina orientalis BB4 receptor. See SEQ ID NO: 1 and 2 in FIG. 1. As will be set forth below, functional studies showed that frog BB4 receptor potently responded to bombesin, suggesting that this new receptor represents the prototype receptor for the bombesin/ranatensin branch of the bombesin-like peptides and is different from the BRS-3 receptor which does not respond to bombesin.

The cDNA encoding the frog BB4 receptor was then used to screen a monkey brain cDNA library (purchased from Clontech, Inc., Palo Alto, Calif.) at low stringency (25% formamide, 5×SSC, 37° C. with washing at 50° C. in 2×SSC) Multiple hybridizing clones were isolated. Sequence analysis of the clones revealed two subtypes with partial sequences: monkey BB4 (SEQ ID NO: 3) and monkey BB5 (SEQ ID NO: 5), which encode SEQ ID NO: 4 and SEQ ID NO: 6, respectively. Monkey BB4 appears highly homologous to frog BB4, i.e., an 88.1 % homology in the 84 amino acid overlap. SEQ ID NO: 3, 4, 5, 6, 11 and 12 are shown below:

```
SEQ ID NO: 3
CAGACATCTG ACGCGGTGTT GAAGACGTGC GGCAAAGCTG TTTGTGTTTG
GATTATCTCC ATGCTACTTG CTGCCCCTGA GGCAGTGTTT TCGGATTTGT
ATGAATTCAC CAGCCCTGAC AAGAATATGT CCTTCAAAAC ATGTGCCCCT
TATCCTGTTT CTGAAkAGCT ACTGCAAGAG ACACATTCGC TGATGTGCTT
CTTAGTGTTC TATATTATTC CCTTGTCTAT TATCTCCGCC TACTACTTCC TC
SEQ ID NO: 4
Gln Thr Ser Asp Ala Val Leu Lys Thr Cys Gly Lys Ala Val Cys
Val Trp Ile Ile Ser Met Leu Leu Ala Ala Pro Glu Ala Val Phe
Ser Asp Leu Tyr Glu Phe Thr Ser Pro Asp Lys Asn Met Ser Phe
Lys Thr Cys Ala Pro Tyr Pro Val Ser Glu Lys Leu Leu Gln Glu
Thr His Ser Leu Met Cys Phe Leu Val Phe Tyr Ile Ile Pro Leu
Ser Ile Ile Ser Ala Tyr Tyr Phe Leu
SEQ ID NO: 5
CAGACCTCAG ATGCTGTGCT GAAGACCTGT GCCAAAGCTG GTGGCATCTG
GATCATGGCT ATGATATTTG CTCTGCCAGA GGCTATATTC TCAAATGTAT
ACACTTTCCA AGGTCCTAAC AGAAACGTAA CATTTGAATC CTGTAACTCC
TACCCTATCT CTGAGAGGCT TTTGCAGGAA ATACATTCTC TGTTGTGTTT
CTTGGTGTTC TACATTATCC CGCTCTCGAT TATCTCCGCC TATTACTTCC
SEQ ID NO: 6
Gln Thr Ser Asp Ala Val Leu Lys Thr Cys Ala Lys Ala Gly Gly
  Ile Trp Ile Met Ala Met Ile Phe Ala Leu Pro Glu Ala Ile Phe
  Ser Asn Val Tyr Thr Phe Gln Gly Pro Asn Arg Asn Val Thr Phe
  Glu Ser Cys Asn Ser Tyr Pro Ile Ser Glu Arg Leu Leu Gln Glu
  Ile His Ser Leu Leu Cys Phe Leu Val Phe Tyr Ile Ile Pro Leu
  Ser Ile Ile Ser Ala Tyr Tyr Phe
SEQ ID NO: 11
CATGTCTCAAAGGCAGCCTCAGTCACCTAATCAGACTTTAATTTCCATTACAAATGACAC
AGAAACATCAAGCTCTGCCGTCTCCAACGATACTACACCTAAAGGATGGACCGGAGACAA
CTCTCCAGGAATAGAAGCACTGTGTGCCATCTATATCACTTATGCTGTGATCATTTCAGT
GGGCATCCTCGGAAATGCTATCCTCATCAAAGTCTTTTTCAAGACTAAATCCATGCAAAC
AGTTCCAAATATTTTCATCACCAGCCTGGCTTTTGGAGATCTGTTACTCCTGCTGACTTG
TGTGCCAGTGGATGCAACCCACTACCTGGCAGAgGGATGGCTGTTTGGAAAGGTCGGTTG
TAAAGTGCTTTCCTTCATCCGGCTCACTTCTGTCGGTGTATCAGTGTTCACGCTAACAAT
TCTCAGCGCTGACAGATACAAAGCAGTCGTGAAGCCACTTGAACGACAGCCCTCCAATGC
CATTCTGAAGACCTGTGCCAAAGCTGGTGGCATCTGGATCATGGCTATGATATTTGCTCT
GCCAGAgGCTATATTCTCAAATGTATACACTTTCCAAGATCCTAACAGAAACGTAACATT
TGAATCCTGTAACTCCTACCCTATCTCTGAGAGGCTTTTGCAGGAAATACATTCTCTGTT
GTGTTTCTTGGTGTTCTACATTATCCCGCTCTCGATTATCTCTGTCTATTATTCTTTGAT
TGCCAGGACTCTTTACAAAAGCACCTTGAACATACCGACTGAGGAACAAAGCCATGCCCG
AAAGCAGATTGAATCCCGGAAGAGAATTGCCAAAACGGTACTGGTGCTGGTGGCTCTGTT
CGCACTCTGCTGGTTGCCGAATCACCTCCTGTATCTCTATCACTCATTCACTTATGAAAG
CTACGCAGAGCCTTCTGATGTCCCTTTCGTTGTCACCATTTTCTCTCGGGTGCTGGCTTT
CAGTAATTCCTGCGTGAACCCCTTTGCTCTGTATTGGCTGAGCAAGACCTTCCAGAAGCA
TTTTAAGGCTCAGCTCTGCTGCTTCAAGGCAGAGCAGCCTGAGCCTCCTCTTGGTGACAC
CCCCCTTAACAACCTCACTGTGATGGGGCGGGTTCCAGCTACTGGGAGTGCACACGTCTC
TGAAATTAGCGTGACCCTGTTTAGTGGCAGTACTGCCAAGAAAGGAGAGGACAAAGTTTA
GATGTTCCATTTAAAATGATTTTAAGTCTCACTGTGTGTATCCAGCTTTAAGCTGTGCAT
AATACGATGTCTTGGCTTTTTGTTGTTCATGAATTGTGTTGAAATTTCCAGAAAGAATTA
TCCTTGTAAGTAATAGACAAACCATCATTTTCTTCAAGGTGCAAACAGTAATGCCATTTG
GGTTTTCTAAATACAATGAAGCCCACCAAGTAGAGAAAAACAGGTTTGGAAATACCAGAC
AACTGTGAAGAAGGCTGTATAGAAGGGATGGCAAAGAAACAAGATAAAGGTTTAAAAATT
GAATTCGATCCCATCTCTCTGATGCGCCTCATCTCTCATCCACTTATGTCGTTGTCATAA
AATAGTGTTTGTGTTTATTTGTGCTGTAAATTTAGGGATTCTACTCTGAAGATCAAAAAG
AGAGAAAACATCTCTTGCAGTCTTTTCTAAAATATCTATCTATCTATCTATCTATGCTTT
CACAGAGTGATTGCCCTTTTCCTTCAATTGCCATTCCATTATAAGAAATCTTACAATTCA
AACAAAAATGTTTTAAGAGTTGGAGGGTATTTAGCGCTGAGTCTGGGGCAGGGTGCTTCC
CTTACTGGGTCAACATGTCCTGAATTTATGTAATTCTACTGCCATTTGATTAATAACTAG
GCAAAGCCATGTCTCACAAGTGTTGTGAGGAATTTATCTTATTCACAGGACGACCAGGGC
CCCAGAGGAGATTTTTTTGTCCAAGTTTCTAAGCTGTCACTGTCAGAAaTTCTTTGCTCT
AAGGAAACTCATTTCATTTGGGTGAGCAGTGCCTTCTTTCTAAAACCCTGAGGAACTGCC
ATGTGACAGCTTCAGCACCTGGCTGTGCGAGCACCATCAGATCATCTACCTGCTTGATTT
TCCTCAACTCGATGGGTGCCTAAGTCCTAGAGCTCACCTCTGTTTCTAGAAGTCAGACAG
GAGGCTCTGACTCTCTATCCTTATCTCCAGCTTGCTAACACAAATGAGCTTGTTAATAAC
TGTAATTCCCAAAACAAAATCCTTCTACTCAATAGATTTGTCACACTTCTGTTTGTGCTT
TCAGAAAATGTATTCTTGAAGTAAATAGAAGCTTACCTTTGGCACTATAGTTACATTCGT
ATTTTTAAAAACTTGATTTTTTTCTTTTAGAGTGCTTTTGGTATTTTTGAGCTTTAAATG
GCTTGTGCCGTTGTGAACTTCTGCTAATTATGTGGCTCAAAAGGGGCGGGGCATACATCA
ACACTGAGCTTTAAATGTCCTTACAGTTGGCTTACTTAAAGTGGGGGTGCTAATATTGTA
TTAACATTGCTTTAAAGTGAGCAAAATTGTTGTAAAAAATCTGGAGGTGAGCAAATAAAT
AAAAAATAAAGTATG- 2595bases
```

Sequence of the cDNA encoding rat BRS-3 receptor (The first codon, ATG (methionine)₂₋₄ is underlined, and the stop codon, TAG₁₂₁₈₋₁₂₂₀, after the last amino acid codon is also underlined.

SEQ ID NO: 12
MSQRQPQSPNQTLISITNDTETSSSAVSNDTTPKGWTGDNSPGIEALCAIYITYAVIISV
GILGNAILIKVFFKTKSMQTVPNIFTTSLAFGDLLLLLTCVPVDATHYLAEGWLFGKVGC
KVLSFIRLTSVGVSVFTLTILSADRYKAVVKPLERQPSNAILKTCAKAGGIWIMAMIFAL
PEAIFSNVYTFQDPNRNVTFESCNSYPISERLLQELHSLLOFLVFYIIPLSIISVYYSLI
ARTLYKSTLNIPTEEQSHARKQIESRKRIAKTVLVLVALFALCWLPNHLLYLYHSFTYES
YAEPSDVPFVVTIFSRVLAFSNSCVNPFALYWLSKTFQKHFKAQLCCFKAEQPEPPLGDT
PLNNLTVMGRVPATGSAHVSEISVTLFSGSTAKKGEDKV-399

The predicted amino acid sequence of the rat BRS-3 receptor clone. (Amino acid code used—Alanine, A; Arginine,R; Asparagine, N; Aspartic acid, D; Cysteine, C; Glutamine, Q; Glutamic acid, E; Glycine, G; Histidine, H; Isoleucine, I; Leucine, L; Lysine, K; Methionine, M; Phenylalanine, F; Proline, P; Serine, S; Threonine, T; Tryptophan, W; Tyrosine, Y; Valine, V)

Analysis of the Topology of the Rat BRS-3 Receptor

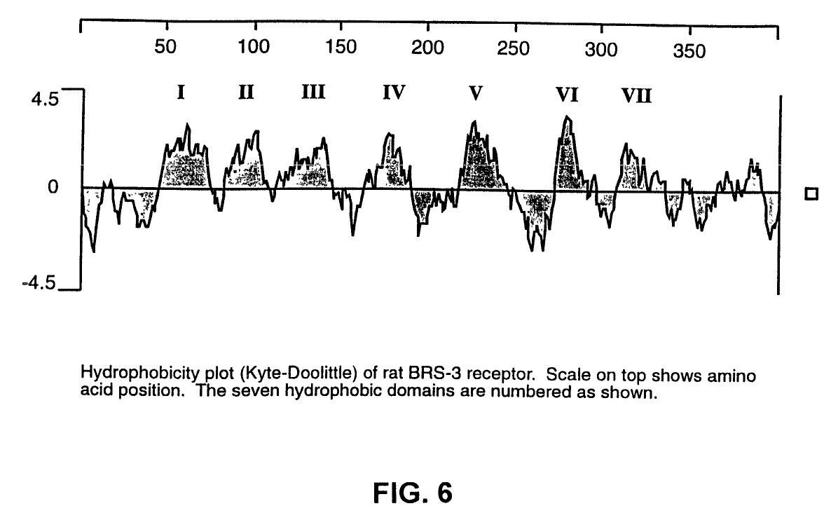

The sequence of the rat BRS-3 receptor was subjected to hydropathy analysis according to the method described in Kyte, J. and Doolittle, R. F., J. Molec. Biol., 1982, 157, pp. 105-132, the contents of which are incorporated herein by reference. The analysis revealed the presence of seven transmembrane region (TMI-VII) (FIG. 6), which is typical for this family of G-protein coupled receptors.

Function Studies (Changes in Intracellular Calcium)

To prepare the receptor RNA for injection into Xenopus oocytes, the linearized cDNA encoding frog BB4 or rat BRS-3 receptor, was phenol extracted, ethanol precipitated, and then transcribed with T7 or T3 RNA polymerase. Transcription reactions were carried out in a 50-100 μl volume containing 5-20 μg DNA template, 40 mM Tris (pH 7.9), 7 mM MgCl₂, 10 mM DTT, 2 mM spermidine, 10 mM NaCl, 25 μg/ml BSA, 0.5 mM ATP, 0.5 mM UTP, 0.5 mM CTP, 0.2 mM GTP, 1 mM 7-Me GpppG (Pharmacia, Piscataway, N.J.), 50-100 units RNA polymerase and 125-250 units RNasin (Promega, Madison, Wis.). The reactions were incubated at 40° C. for 90 minutes, treated with FPLC purified DNase (Pharmacia, Piscataway, N.J.), phenol extracted twice, ethanol precipitated twice, and then resuspended in 5-10 μl H₂O. See Julius, et al. Science 1988; 241:558-564, which is hereby incorporated by reference.

To measure bombesin- and bombesin-like peptide (Wu, J. M., et al., Mol. Pharmacol., 1996, 50, 1355-1363; and Pradham, T. K., et al., Eur. J. Pharmacol., 1998, 343, pp. 273-287, the contents of both of which are incorporated herein by reference) induced changes in intracellular calcium, the procedure described in Sandberg, et al. FEBS Lett 1988; 241:177-180 (the contents of which are hereby incorporated by reference) was followed with some modifications (see Spindel, et al., Mol. Endocrinol. 1990; 4:1956-1963; and Giladi, et al., Biotechniques 1991; 10:744-747), the contents of both of which are hereby incorporated by reference). More specifically, oocytes were removed from an albino Xenopus, treated with collagenase, defollicated, and then injected in the presence of OR-2 (a buffer solution suitable for frog oocytes) without calcium. The injection needles were rinsed with 1 mM EDTA before each use. For injection, the transcribed RNA (typically, 1-2 μl) was dried down and then suspended in an equal volume of an aequorin solution. The aequorin solution was prepared at a concentration of 1 mg/ml in 1 mM EDTA and stored in aliquots at −85° C. Aequorin was obtained from Friday Harbor Photoproteins, Friday Harbor, Wash.

To record the bombesin-induced response, oocytes were placed in 500 μl OR-2 in 12×55 mm disposable polystyrene tubes in a luminometer. Light output from the oocyte as recorded by the luminometer is a function of ligand-induced increases in intracellular calcium. The baseline response to OR-2 was first recorded, followed by the recording of the response to bombesin.

As a positive control, Xenopus oocytes containing exogenous human GRP receptor were also prepared and assayed in analogous manners.

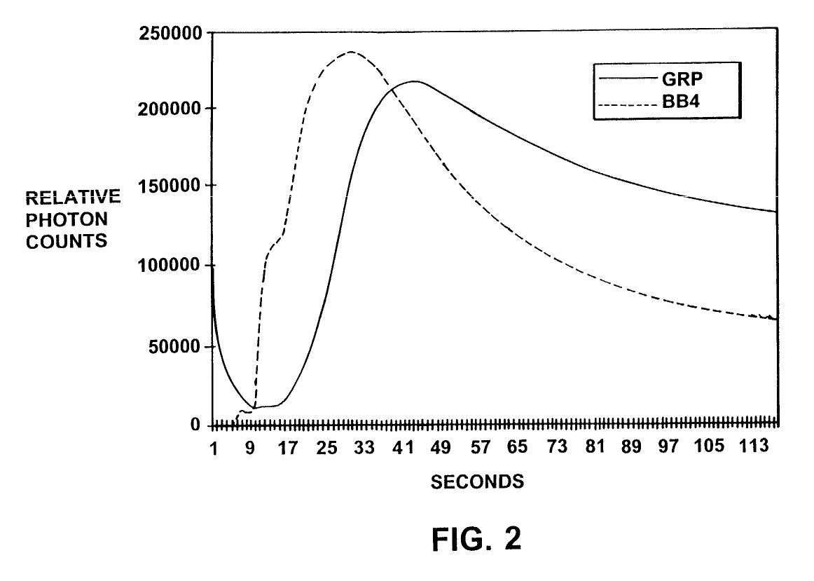

FIG. 2 demonstrates the respective responses of GRP and BB4 receptors to 1 nM (in the OR-2 buffer) of bombesin. It is clear that the BB4 receptor, unlike the BRS-3 receptor, potently responded to bombesin.

### OTHER EMBODIMENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

For example, contemplated equivalents of this invention include nucleic acid or peptide sequences which are substantially identical to those clearly described above and explicitly claimed below. By “substantially identical” is meant a nucleic acid or peptide exhibiting at least 50%, preferably 85%, more preferably 90%, and most preferably 95% homology to a reference amino acid or nucleic acid sequence. For peptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

Sequence identity is typically measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications. Conservative substitutions typically include substitutions within the following groups: (i) glycine, alanine; (ii) valine, isoleucine, leucine; (iii) aspartic acid, glutamic acid, asparagine, glutamine; (iv) serine, threonine; (v) lysine, arginine; and (vi) phenylalanine, tyrosine.

Furthermore, nucleic acids and peptides which are allelic variations, natural mutants, and induced mutants are also within the scope of this invention.

Still other contemplated equivalents of this invention include peptides which are shorter than a receptor of this invention (e.g., a fragment thereof) which has at least one antigenic determinant that is immunologically reactive with an antigenic determinant of that receptor.

Other embodiments are also within the claims set forth below.

What is claimed is:

1. A pure nucleic acid encoding a receptor for a bombesin-like peptide, said receptor comprising SEQ ID NO: 12.

2. The nucleic acid of claim 1, wherein said nucleic acid is genomic DNA.

3. The nucleic acid of claim 1, wherein said nucleic acid is cDNA.

4. The nucleic acid of claim 1, wherein said nucleic acid is RNA.

5. A vector comprising the nucleic acid of claim 1 for expression of the receptor encoded by said nucleic acid.

6. A cell into which the nucleic acid of claim 1 has been introduced.

7. A purified nucleic acid which hybridizes to the full length complement of the DNA sequence as set forth in SEQ ID NO: 11 under high-stringency hybridization conditions; and encodes a receptor protein for a bombesin-like peptide, wherein said receptor protein is expressed in the tissues of vertebrates.

8. A vector comprising the nucleic acid of claim 7 for expression of the receptor encoded by said nucleic acid.

9. A cell into which the nucleic acid of claim 7 has been introduced.

10. The nucleic acid of claim 7, wherein said nucleic acid is genomic DNA.

11. The nucleic acid of claim 7, wherein said nucleic acid is cDNA.

12. The nucleic acid of claim 7, wherein said nucleic acid is RNA.

13. A method of screening for a compound which interacts with a receptor protein for a bombesin-like peptide, said method comprising:
providing a host cell transfected with the nucleic acid encoding a receptor for a bombesin-like peptide, comprising the amino acid sequence as set forth in SEQ ID NO: 12;
culturing the host cell under conditions that would allow expression of the receptor protein on the surface of the host cell;
contacting the compound with the receptor protein; and
detecting an interaction between the compound and said expressed receptor protein.

14. A pure receptor protein which is encoded by a nucleic acid including SEQ ID NO: 2, SEQ ID NO: 4, or SEQ ID NO: 6 and binds to a bombesin-like peptide.

15. A pure receptor protein which is encoded by a nucleic acid that hybridizes to a DNA sequence under a high-stringency hybridization condition and which binds to a bombesin-like peptide, said DNA sequence being selected from the group consisting of SEQ ID NO: 1, SEQ ID NO: 3, and SEQ ID NO: 5.

16. A pure receptor protein which is encoded by a nucleic acid that hybridizes to a DNA sequence under a low-stringency hybridization condition and which binds to a bombesin-like peptide, said DNA sequence being selected from the group consisting of SEQ ID NO: 1, SEQ ID NO: 3, and SEQ ID NO: 5.

17. A pure antibody which is specific for the protein of claim 14.

18. A pure antibody which is specific for the protein of claim 15.

19. A pure antibody which is specific for the protein of claim 16.

20. A method of screening for a compound which interacts with a receptor protein for a bombesin-like peptide, said method comprising:
providing a cell which expresses the receptor protein of claim 15;
contacting the compound with said expressed receptor protein; and
detecting an interaction between the compound and said expressed receptor protein.

21. A method of screening for a compound which interacts with a receptor protein for a bombesin-like peptide, said method comprising:
providing a cell which expresses the receptor protein of claim 16;
contacting the compound with said expressed receptor protein; and
detecting an interaction between the compound and said expressed receptor protein.
